(19) United States
(12) Patent Application Publication
(10) Pub. No.: US 2003/0165898 A1
(43) Pub. Date: Sep. 4, 2003

(54) Method for concurrent amplification and real time detection of polymorphic nucleic acid sequences

(76) Inventor: Alison Todd, Glebe New South Wales (AU)

Correspondence Address: BROWDY AND NEIMARK, P.L.L.C., 624 NINTH STREET, NW, SUITE 300, WASHINGTON, DC, 20001-5303, US

(21) Appl. No.: 10/169,575

(22) PCT Filed: Jan. 5, 2001
(86) PCT No.: PCT/AU01/00008

(30) Foreign Application Priority Data
Jan. 5, 2000 (AU) PQ 4957

(51) Int. Cl. C12Q 1/68; C12P 19/34

(52) U.S. Cl. 435/6; 435/91.2

(57) ABSTRACT

The present invention provides a method of detecting a genetic polymorphism in an individual or between individuals. The method comprises the following steps, (1) obtaining a sample containing nucleic acid from an individual; (2) contacting the sample, under conditions which permit primer-initiated nucleic acid amplification and nucleic acid cleavage, with (i) a primer suitable for initiating amplification, (ii) an indicator system which provides a signal proportional to the amount of amplification product, and (iii) a sequence specific nucleic acid cleavage agent; and (3) measuring the signal produced by the indicator system against time. Cleavage of the amplification product by the cleavage agent results in an inhibition of the rate of accumulation of amplification product comprising the sequence recognised by the cleavage agent relative to the rate of accumulation of amplification product not comprising the sequence recognised by the cleavage agent.

### FIELD OF INVENTION

The present invention relates to methods for detecting a genetic polymorphism in an individual, or between individuals. In particular the invention relates to methods that employ a sequence specific nucleic acid cleavage agent, to inhibit amplification of specific nucleic acid sequences. The invention can also use real time analysis to determine whether the sequence specific nucleic acid cleavage agent either has, or lacks, the ability to temporally inhibit or delay amplification due to the presence or absence of a specific allele in a target nucleic acid.

### BACKGROUND OF THE INVENTION

Genetic Sequences as Markers of Disease

A variety of inherited and acquired diseases are associated with genetic variations such as point mutations, deletions and insertions. Some of these variations are directly associated with the presence of disease, while others correlate with disease risk and/or prognosis. There are more than 500 human genetic diseases that result from mutations in single genes (Antonarakis, 1989; Watson et al., 1983). These include cystic fibrosis, haemocromatosis, muscular dystrophy, α1-antitrypsin deficiency, phenylketonuria, sickle cell anaemia or trait, and various other haemoglobinopathies (Antonarakis, 1989; Watson et al., 1983). The prognosis for individuals that are homozygous, as opposed to heterozygous, for such mutations is frequently different. Analysis of inherited diseases is therefore most conveniently performed using assays that simultaneously give information about the presence or absence of both mutant and wild type alleles in a single reaction.

Cancer is thought to develop due to the accumulation of genetic lesions in genes involved in cellular proliferation or differentiation. The ras proto-oncogenes, K-ras, N-ras and H-ras, and the p53 tumour suppressor gene are examples of genes that are frequently mutated in human cancers. Specific mutations in these genes lead to an increase in transforming potential. Aberrant patterns of methylation, frequently associated with regulatory regions of specific genes, can also serve as markers for tumours cells. Genetic analysis may have application in the clinic for assessing disease risk, diagnosis of disease, predicting a patient's prognosis or response to therapy, and monitoring a patient's progress. Genetic tests for analysis of genetic alterations associated with cancer must be highly sensitive since the tumour cells (and their associated mutations) are often present in a large background of non-tumour cells in clinical specimens. The introduction of genetic tests for oncology will depend on the development of sensitive, simple, inexpensive, and rapid assays for genetic variations.

Method for in vitro Amplification of Nucleic Acids

Methods of in vitro nucleic acid amplification have wide-spread applications in genetics, disease diagnosis and forensics. Many techniques for amplification of known nucleic acid sequences (“targets”) have been described. These include assays mediated by DNA polymerase, such as the polymerase chain reaction (“PCR”) (U.S. Pat No. 4,683,202; U.S. Pat. No. 4,683,195; U.S. Pat. No. 4,000,159; U.S. Pat No. 4,965,188; U.S. Pat. No. 5,176,995; Chehab et al., 1987; Saiki et al., 1985; Walder et al., 1993) and the strand displacement amplification assay (“SDA”) (Walker et al., 1992), as well as assays mediated by RNA polymerase which are known as transcription-mediated amplification (“TMA”) (Jonas et al., 1993), self-sustained sequence replication (“3SR”) (Gingeras et al., 1990) and nucleic acid sequence replication based amplification (“NASBA”) (Compton, 1991). The amplification products (“amplicons”) produced by PCR and SDA are DNA, whereas RNA amplicons are produced by TMA, 3SR and NASBA. The DNA or RNA amplicons generated by these methods can be used as markers of nucleic acid sequences associated with specific disorders. DNA or RNA templates can be analyzed for the presence of sequence variation (i.e. mutations) associated with disease.

Polymerase Chain Reaction (PCR)

The polymerase chain reaction (PCR) is a powerful, exquisitely sensitive procedure for in vitro amplification of specific segments of nucleic acids (Chehab et al., 1987; Saiki et al., 1985; U.S. Pat No. 4,683,202; U.S. Pat. No. 4,683,195; U.S. Pat. No. 4,800,159; U.S. Pat. No. 4,965,188; U.S. Pat. No. 5,176,995). The PCR is mediated by oligonucleotide primers that flank the target sequence to be synthesized. Production of amplicons occurs as a result of temperature cycling (thermocycling). Template DNA is first denatured by heating, the reaction is then cooled to allow the primers to anneal to the target sequence, and then the primers are extended by DNA polymerase. The cycle of denaturation, annealing and DNA synthesis is repeated many times and the products of each round of amplification serve as templates for subsequent rounds. This process results in the exponential amplification of amplicons which incorporate the oligonucleotide primers at their 5′ termini and which contain newly synthesized copies of the sequence located between the primers.

The PCR is extremely versatile and many modifications of the basic protocol have been developed. Primers used for the PCR may be perfectly matched to the target sequence or they can contain mismatched and/or modified bases. Additional sequences at the 5′ end of primers can facilitate capture of PCR amplicons and the inclusion of labelled primers can facilitate detection. The inclusion of mismatched bases within primers can result in the induction of new restriction enzyme (RE) recognition/cleavage sites (Cohen and Levinson 1988; Todd et al., 1991a; Todd et al., 1991b; WO 96/32500) or in the induction of new deoxyribozyme (DNAzyme) recognition/cleavage sites (WO 99/50452). The recognition sites for these enzymes can span a sequence which lies partially within the primer and partially within the newly synthesized target sequence. The general rules for designing primers which contain mismatched bases located near the 3′ termini have been established (Kwok et al., 1990).

Enzymes Which Cleave Nucleic Acids

Restriction enzymes (REs) are catalytic proteins that cleave DNA at specific recognition sequences, typically four to eight base pairs (bp) in length. They have been used extensively in combination with in vitro amplification for analysis of small sequence variations including detection of point mutations. One method, known as restriction fragment length polymorphism (RFLP), involves ascertaining whether a RE site is present or absent at the locus of interest. In rare instances mutations can be detected if they happen to lie within a naturally occurring RE recognition/cleavage site.

WO 84/01389 describes a method for discriminating between wild type genes and non wild type variants by screening for the presence or absence of RE sites. The inclusion of mismatched bases within primers used to facilitate in vitro amplification can result in the induction of artificial RE recognition sites. This strategy has increased the number of loci which can be analysed by RFLP (Cohen and Levinson, 1988) and related protocols, such as enriched PCR (Levi et al., 1991; Todd et al., 1991a) and REMS-PCR (see below) which also depend on the presence or absence of RE sites in amplification products.

Catalytic nucleic acid molecules can also cleave nucleic acids. As used herein, catalytic nucleic acid molecule means a catalytic DNA molecule (also known in the art as a deoxyribozyme or DNAzyme) or a catalytic RNA molecule (also known in the art as a ribozyme) which specifically recognizes and cleaves a distinct target nucleic acid sequence. Catalytic DNA molecules have been shown to be capable of cleaving both RNA (Breaker and Joyce, 1994; Santoro and Joyce, 1997) and DNA (Carmi et al., 1996) molecules. Similarly, catalytic RNA molecules (ribozymes) have been shown to be capable of cleaving both RNA (Haseloff and Gerlach, 1988) and DNA (Raillard and Joyce, 1996) molecules. Catalytic nucleic acid can only cleave a target nucleic acid sequence provided that target sequence meets minimum sequence requirements. The target sequence must be complementary to the hybridizing arms of the catalytic nucleic acid and the target must contain a specific sequence at the site of cleavage. Examples of such sequence requirements at the cleavage site include the requirement for purine:pyrmidine ribonucleotides for cleavage by the 10-23 deoxyribozyme (Santoro and Joyce, 1997), and the requirement for the sequence uridine:X where X can equal A, C or U but not G, for hammerhead ribozymes (Perriman et al., 1992). The 10:23 deoxyribozyme is a deoxyribozyme that is capable of cleaving nucleic acid substrates at specific RNA phosphodiester bonds (Santoro and Joyce, 1997, Joyce, 2000). This deoxyribozyme has a catalytic domain of 15 deoxynucleotides flanked by two substrate-recognition domains (arms).

Catalytic nucleic acid molecules have be exploited in vitro to distinguish between targets that differ by as little as a single point mutation (WO 99/50452, Cairns et al., 2000). This is achieved by targeting a specific sequence that is present in wild-type but not mutant templates or vice versa. Catalytic nucleic acid can be used to analyse the products of in vitro amplification mediated by a variety of techniques including the PCR and TMA. Deoxyribozymes are well suited for use in combination with PCR since, unlike the majority of protein enzymes, they are not irreversibly denatured by exposure to high temperatures during the denaturation step of PCR. When deoxyribozymes are used in combination with the PCR, chimeric DNA/RNA primers can be used to introduce purine:pyrimidine ribonucleotide residues into the amplicons to create sites that could potentially be cleaved by a deoxyribozyme. Furthermore, if the target sequence does not contain a natural purine:pyrimidine sequence, the cleavage site for the deoxyribozyme can be induced using mismatched primers in the same way that mismatched primers have been used to induce artificial RE sites (WO 99/50452). The chimeric primers hybridize to the target sequence adjacent to polymorphic region that is being analysed. deoxyribozymes present in the PCR mix are designed to cleave PCR amplicons provided the sequences of the deoxyribozyme hybridizing arms are fully complementary to the PCR amplicons. Sequence variations at the locus being examined, which result in mismatches between the amplified region and the 5′ hybridizing arm of the deoxyribozyme, can disrupt deoxyribozyme cleavage. Analysis of the fragments generated by deoxyribozyme cleavage allows ascertainment of the sequence at the locus being examined.

Real-Time Homogeneous Amplification and Detection

Several methods allow simultaneous amplification and detection of nucleic acids in a closed system, i.e., in a single reaction system. These methods include the Molecular Beacon (Tyagi and Kramer, 1996), Taqman™(Lee et al., 1993; Livak et al., 1995), and HybProbe assays (Wittwer et al., 1997) which depend on internal hybridization probes, and the Sunrise™(Nazarenko et al., 1997) and DzyNA assays (WO 99/45146) which are mediated by modified primers. All of these approaches have been used to detect the products of PCR and some of the strategies have been linked to other amplification technologies. For example, Molecular Beacon probes have also been used to detect the products of NASBA (Leone et al., 1998) and DzyNA primers are also compatible with SDA and TMA (WO 99/45146).

Sealed reaction formats have several advantages over methods that separately analyze amplicons following amplification reactions. Closed system methods are faster and simpler because they require fewer manipulations. A closed system also eliminates the potential for false positives associated with contamination by amplicons from other reactions. Homogeneous reactions can be monitored in real time, and changes in the signal intensity indicate amplification of a specific nucleic acid sequence present in the sample.

REMS-PCR (Restriction Endonuclease Mediated Selective PCR)

REMS-PCR provides a sensitive, rapid and reliable method that is suitable for analysis of genetic variations that are associated with disease (WO 96/32500; Ward et al., 1998; Fuery et al., 2000). REMS-PCR can be used for the analysis of either acquired or inherited genetic polymorphisms (eg point mutations, small deletions or insertions). REMS-PCR facilitates selective amplification of variant sequences in reactions that contain all reagents, including all enzymes, at the initiation of the PCR. The assay requires concurrent activity of a RE and a DNA polymerase. In this protocol the inclusion of the thermostable RE in the PCR results in (i) inhibition of amplification of sequences which contain the recognition site for the specific RE; and (ii) selective amplification of sequences which lack the recognition site.

The RE and the polymerase must i) function in identical reaction conditions (eg., salt, pH) which must be compatible with the PCR and ii) must be sufficiently thermostable in these reaction conditions to retain activity during the thermocycling which is required for the PCR. REs which are suitable for combination with the PCR must be active at temperatures which are compatible with stringent conditions for annealing of primers during the PCR, typically 50° C.-65° C. The RE recognition site may be either natural or PCR-induced and must span the nucleotide bases that are being analysed.

Controls can be included in reactions to confirm that the reaction conditions, including the amount of template DNA, are adequate for amplification by the PCR. PCR control primers can flank any region that does not contain the RE recognition/cleavage site. The presence of PCR control amplicons allows confirmation that all the reaction components and conditions were adequate for the PCR. A second control can be included to confirm that the RE mediates inhibition of amplification by the PCR. RE control primers induce the recognition/cleavage site for the RE used in the REMS-PCR protocol.

In previously published protocols where REMS-PCR was monitored by gel electrophoresis, the reactions had to be stopped at a time point before diagnostic amplicons containing wild type sequences and RE control amplicons become visible. This meant that a standard number of amplification cycles, typically 30 cycles, were performed and analysis of samples by electrophoresis could only provide a yes/no answer as to the presence or absence of a particular sequence (mutation) at the diagnostic locus. This made the technology less well suited to the analysis of inherited genetic disorders since identification of a heterozygous individual required two REMS-PCR reactions (one targeting the mutant allele and a second targeting the wild type allele). When REMS-PCR and electrophoresis was being used for analysis of rare mutations, the necessity to stop the reaction at a set number of cycles meant that there was the potential for false negatives when the mutant template was present in very low abundance. This was particularly a problem with certain types of clinical specimen where there was only limited amount of nucleic acid template or when the template was of poor integrity. In these instances, amplification of the rare mutant alleles would not always reach the threshold whereby they are detectable in the standard number of cycles of amplification chosen for the assay. In these cases results could be obtained in a proportion of cases by re-analysing the samples using larger numbers of cycles. Conversely, when the template was present in excess and/or the efficiency of the RE was sub-optimal, the appearance of RE control amplicons means that results could not be immediately interpreted. The reactions had to be subjected to post-PCR manipulation (eg further digestion with the RE used in the REMS-PCR or sequencing) or the sample had to be re-analysed using a lesser number of cycles.

The present invention provides improved methods for detecting a genetic polymorphism in an individual, or between individuals. The present invention also allows for the real time analysis of a polymorphism, and can be performed in a single closed reaction vessel.

### SUMMARY OF INVENTION

In a first aspect the present invention consists in a method of detecting a genetic polymorphism in an individual or between individuals, the method comprising the following steps:

(1) obtaining a sample containing nucleic acid from an individual;

(2) contacting the sample, under conditions which permit primer-initiated nucleic acid amplification and nucleic acid cleavage, with

(i) a primer suitable for initiating amplification,

(ii) an indicator system which provides a signal proportional to the amount of amplification product, and

(iii) a sequence specific nucleic acid cleavage agent; and

(3) measuring the signal produced by the indicator system against time;

wherein cleavage of the amplification product by the cleavage agent results in a delay in the accumulation of amplification product comprising the sequence recognised by the cleavage agent relative to the accumulation of the amplification product not comprising the sequence recognised by the cleavage agent.

In a preferred embodiment the primers are designed such that they induce the sequence recognised by the sequence specific nucleic acid cleavage agent into the nucleic acid resulting from amplification of the sample nucleic acid not including the polymorphism or into the nucleic acid resulting from amplification of the sample nucleic acid including the polymorphism.

In a further preferred embodiment the sequence specific nucleic acid cleavage agent is a thermostable restriction endonuclease, preferably selected from the group consisting of Bst NI, Bsl I, Tru 9I, Tsp 509 I, Tsp 45 I, Tth 111 I, Tsp RI, Tse I, Tfi I, Sml I, Bso B I, Bst E II, Bst F5 I, Psp G I and Sfi I.

In an alternative embodiment the sequence specific nucleic acid cleavage agent is a catalytic nucleic acid, preferably either a ribozyme or a deoxyribozyme. It is further preferred that at least one primer comprises a region which binds to the sample nucleic acid and a region which is an antisense sequence of the catalytic nucleic acid such that on amplification the catalytic nucleic acid is produced.

In another preferred embodiment the signal produced by the indicator system is fluorescence. It is further preferred that the indicator system comprises a catalytic nucleic acid and a substrate, the substrate comprising a fluorophore and a molecule that quenches fluorescence from the fluorophore separated by a site cleavable by the catalytic nucleic acid, wherein the primers are designed such that the amplification products comprise the catalytic nucleic acid.

In this embodiment it is preferred that one primer comprises a region which binds to the nucleic acid and a region which is an antisense sequence of the catalytic nucleic acid.

The indicator system may be any of a number of such systems well known in the art, eg TaqMan™, Molecular Beacon™, Hybidisation Probe (Roche), and Sunrise™.

It is preferred that the sample nucleic acid is a DNA molecule, and where the sample nucleic acid is an RNA molecule, step (2) further comprises the step of first reverse transcribing the RNA sequence to DNA.

The amplification methodology may be any of a number of such systems well known in the art, eg polymerase chain reaction (PCR), strand displacement amplification assay (SDA), transcription-mediated amplification reaction (TMA), self-sustained sequence replication amplification reaction (3SR), and nucleic acid sequence replication based amplification reaction (NASBA), however, PCR is preferred.

In a still further preferred embodiment the genetic polymorphism is within a gene selected from the group consisting of; ras proto-oncogenes (K-ras, N-ras, and H-ras), p53 tumour suppressor gene, a HIV-I gene, haemocromatosis, cystic fibrosis trans-membrane conductance regulator, α-antitrypsin, Factor V and β-globin.

In a second aspect the present invention consists in a method of detecting an epi-genetic polymorphism in an individual or between individuals, the method comprising the following steps:

(1) obtaining a sample containing nucleic acid from an individual;

(2) reacting the nucleic acid from step (1) with a compound that differentially modifies nucleotide bases depending on whether the specific base contains, or lacks, a covalent modification;

(3) contacting the nucleic acid from step (2), under conditions which permit primer-initiated nucleic acid amplification and nucleic acid cleavage, with

(i) a primer suitable for initiating amplification,

(ii) an indicator system which provides a signal proportional to the amount of amplification product, and

(iii) a sequence specific nucleic acid cleavage agent; and

(4) measuring the signal produced by the indicator system against time;

wherein cleavage of the amplification product by the cleavage agent results in a delay in the accumulation of amplification product comprising the sequence recognised by the cleavage agent relative to the accumulation of the amplification product not comprising the sequence recognised by the cleavage agent.

In a preferred embodiment the primers are designed such that they induce the sequence recognised by the sequence specific nucleic acid cleavage agent into the nucleic acid resulting from amplification of the sample nucleic acid not including the polymorphism or into the nucleic acid resulting from amplification of the sample nucleic acid including the polymorphism.

In another preferred embodiment the covalent modification is methylation of a base and the nucleic acid is reacted with bisulphite.

It is also preferred that the sequence specific nucleic acid cleavage agent is a thermostable restriction endonuclease selected from the group consisting of Bst N I, Psp G I, Bsl I, Tru9 I, Bst U I and Tsp509 I.

In yet another embodiment the epi-genetic polymorphism is within the promoter region of a gene associated with human tumours. It is preferred that the promoter region is from a gene selected from the group consisting of: p16, E-cadherin, the von Hippel Lindau (VHL) gene, BRCA1, p15, hMLH1, ER, HIC1, MDG1, GST-π, O⁶-MGMT, calcitonin, urokinase, S100A4, and myo-D.

In a still further preferred embodiment of each aspect of the present invention the endonuclease activity of the thermostable restriction endonuclease decreases throughout the amplification process.

It is also preferred that the sample is obtained from a mammal, preferably a human. It is also preferred that the method is performed in a closed vessel or chamber.

The method of the present invention can be used for the analysis of a range of genetic polymorphisms including point mutations, small deletions and insertions. Furthermore, the present invention allows for analysis of inherited polymorphisms by facilitating simultaneous detection of both the homozygous and heterozygous states in a single reaction. In addition, when acquired mutations are being analysed, the present invention abrogates the need to terminate the reactions prematurely before rare mutant alleles have amplified and this is likely to increase the sensitivity and reliability of the assay.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### DETAILED DESCRIPTION

The invention will hereinafter be described by way of the following non-limiting Figures and Examples.

### BRIEF DESCRIPTION OF THE ACCOMPANYING DRAWINGS

FIG. 1 provides the results obtained from the experiment outlined in the Example section.

Unless otherwise indicated, the recombinant DNA techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (Editors), Current Protocols in Molecular Biology, Greene Pub. Associates and Wiley-Interscience (1988, including all updates until present) and are incorporated herein by reference. These techniques include: methods for isolating nucleic acid molecules, including, for example, phenol chloroform extraction, quick lysis and capture on columns (Kramvis et al., 1996; Liu et al., 1995; Sambrook et al., 1989; U.S. Pat. No. 5,582,988); methods of detecting and quantitating nucleic acid molecules; methods of detecting and quantitating catalytic nucleic acid activity; methods of amplifying a nucleic acid sequence including, for example, PCR, SDA, TMA and 3SR (U.S. Pat No. 4,683,202; U.S. Pat. No. 4,683,195; U.S. Pat. No. 4,000,159; U.S. Pat. No. 4,965,188; U.S. Pat. No. 5,176,995; Chehab et al., 1987; Fahy et al., 1991; Jonas et al., 1993; Saiki et al., 1985; Walder et al., 1993; Walker et al., 1992); methods of designing and making primers for amplifying a particular target sequence; and methods of determining whether a catalytic nucleic acid molecule cleaves an amplified nucleic acid segment including, by way of example, fluorescence resonance energy transfer (FRET) (Cuenoud and Szostak, 1995; WO 94/29481).

The term “individual” is used herein in the broadest sense and is intended to cover human and non-human animals, bacteria, yeast, fungi and viruses. Accordingly, the nucleic acid can be from any organism, and the sample can be any composition containing, or suspected to contain, nucleic acid molecules. In one embodiment, the nucleic acid is from a plant, or from an animal such as, for example, a mouse, rat, dog, guinea pig, ferret, rabbit, and primate. In another embodiment, the nucleic acid is in a sample obtained from a source such as water or soil. In a further embodiment, the target is from a sample containing bacteria, viruses or mycoplasma.

“Amplification” of a nucleic acid sequence refers to a method where polmerase copies target nucleic acid resulting in an increase in the number of copies of the target nucleic acid.

In the instant methods, the nucleic acid amplification can be performed according to any suitable method known in the art, and preferably according to one selected from the group consisting of PCR, SDA, TMA, NASBA, rolling circle amplification and 3SR.

The PCR is an in vitro DNA amplification procedure that requires two primers that flank the target sequence to be synthesized. A primer is an oligonucleotide sequence that is capable of hybridising in a sequence specific fashion to the target sequence and extending during the PCR. Amplicons or PCR products or PCR fragments are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the induction of RE or catalytic nucleic acid recognition/cleavage sites in specific target sequences. Primers may also contain additional sequences and/or modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

The sequence specific nucleic acid-cleavage agent used in the present methods can be any molecule or compound that is able to distinguish and cleave a specific allele of a polymorphic region, but is not able to cleave another allele of the same region. The alleles of the polymorphic region can differ by, for example, a single base mutation (point mutation), or by small insertions or deletions. Preferably, the sequence specific nucleic acid cleavage agent is selected from the group consisting of thermostable restriction endonucleases and catalytic nucleic acids. It is preferred that the catalytic nucleic acid is either a ribozyme or a deoxyribozyme.

In the present invention, the specific nucleic acid cleavage agent and the polymerase must i) function in identical reaction conditions (eg., salt, pH) and ii) be sufficiently thermostable in these reaction conditions to retain activity during the thermocycling which is required for amplification. Sequence specific nucleic acid cleavage agents which are suitable for the present invention are preferably active at temperatures which are compatible with stringent conditions for annealing of oligonucleotide primers during the amplification, typically 50° C.-65° C. Identification of additional reaction conditions that promote the preservation of concurrent cleavage activity during thermocycling for these and/or other thermophilic enzyme combinations can be achieved following routine testing using the activity/thermostability assay using assay techniques such as described in WO96/32500 and Fuery et al. (2000). Similarly, conditions allowing polymerase and catalytic nucleic acid activity can be determined (Impey et al 2000).

The term catalytic nucleic acid refers to a DNA molecule or DNA-containing molecule (also known in the art as a “deoxyribozyme” or “DNAzyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art.

Typically, the catalytic nucleic acid contains an anti-sense sequence for specific recognition of a target nucleic acid, and a nucleic acid cleaving enzymatic activity. The catalytic strand cleaves a specific site in a target nucleic acid. The types of ribozymes that are particularly useful in this invention are the hammerhead ribozyme (Haseloff and Gerlach 1988, Perriman et al., 1992) and the hairpin ribozyme (Shippy et al., 1999).

DzyNA-PCR is a general strategy for the detection of specific genetic sequences associated with disease or the presence of foreign agents (WO 99/45146, Todd et al., 2000). The method provides a system that allows gene amplification coupled with signal detection in a single closed vessel. The strategy involves in vitro amplification of genetic sequences using a DzyNA primer that harbors the complementary (antisense) sequence of a 10:23 deoxyribozyme. During amplification, amplicons are produced which contain active (sense) copies of deoxyribozymes that cleave a reporter substrate included in the reaction mix. Cleavage of this reporter substrate is indicative of successful amplification of the target nucleic acid sequence. The accumulation of amplicons during PCR can be monitored by changes in fluorescence produced by separation of fluoro/quencher dye molecules (eg FAM/TAMRA or FAM/DABCYL) incorporated into opposite sides of a deoxyribozyme cleavage site within a reporter substrate. Real time fluorometric measurements can be performed on the ABI PRISM 7700 Sequence Detection System (SDS) or other platforms that allow monitoring of fluorescence changes in real time.

The ABI PRISM™ 7700 SDS software can be used to report the increase in reporter dye fluorescence (eg FAM fluorescence at 530 nm) following cleavage of a substrate by deoxyribozymes during DzyNA PCR. The cycle threshold value (Ct) is defined as the cycle when fluorescence exceeds a defined baseline signal (threshold ΔRn) within the log phase of PCR product accumulation (Heid et al., 1996). A standard curve can be generated when the log of the starting amount of template is plotted against the Cₜ value. Quantitation of the amount of nucleic acid in reactions can be estimated from the standard curve. Similarly, the ABI PRISM™ 7700 SDS software can be used to report the increase in reporter dye fluorescence following cleavage of the reporter probe by polymerase during TaqMan™PCR.

The general strategy of DzyNA amplification is very flexible. In addition to PCR, it could be incorporated into other strategies for in vitro amplification of nucleic acids (WO 99/45146, Todd et al., 2000). These include strand displacement amplification (SDA) (Walker et al., 1992), which produces DNA products, and transcription-mediated amplification (TMA) Jonas et al., 1993) which produces RNA products. Theoretically, the catalytic nucleic acid molecule encoded by a DzyNA primer could be either a deoxyribozyme if PCR or SDA were used, or a ribozyme if TMA were used to mediate nucleic acid amplification. Furthermore, in vitro evolution technology has facilitated the discovery of deoxyribozymes and ribozymes that are capable of catalyzing a broad range of reactions including cleavage (Breaker 1997; Carmi et al., 1996; Raillard and Joyce, 1996; Santoro and Joyce, 1998) and ligation of nucleic acids (Cuenoud and Szostak, 1995), porphyrin metallation (Li and Sen, 1996), and the formation of carbon-carbon (Tarasow et al., 1997), ester (Illangasekare et al., 1995) or amide bonds (Lohse and Szostak, 1996). Therefore it may be possible to develop systems for detection of in vitro amplification products where the reporter substrate is a molecule other than a nucleic acid and/or the readout of the assay is dependent on a modification other than cleavage of the substrate.

The ‘TaqMan’ fluorescence energy transfer assay uses a nucleic acid probe complementary to an internal segment of the target DNA. The probe is labelled with two fluorescent moieties with the property that the emission spectrum of one overlaps the excitation spectrum of the other; as a result the emission of the first fluorophore is largely quenched by the second. The probe is present during PCR and if PCR product is made, the probe becomes susceptible to degradation via a 5′-nuclease activity of Taq polymerase that is specific for DNA hybridized to template. Nucleolytic degradation of the probe allows the two fluorophores to separate in solution, which reduces the quenching and increases intensity of emitted light from the first fluorophore.

Probes used as Molecular Beacons are based on the principle of single-stranded nucleic acid molecules that possess a stem-and-loop structure. The loop portion of the molecule is a probe sequence that is complementary to a predetermined sequence in a target nucleic acid. The stem is formed by the annealing of two complementary arm sequences that are on either side of the probe sequence. The arm sequences are unrelated to the target sequence. A fluorescent moiety is attached to the end of one arm and a non-fluorescent quenching moiety is attached to the end of the other arm. The stem keeps these two moieties in close proximity to each other, causing the fluorescence of the fluorophore to be quenched by fluorescence resonance energy transfer to the quencher. When the molecular beacon probe encounters a target molecule, it forms a hybrid that is longer and more stable than the hybrid formed by the arm sequences. Since nucleic acid double helices are relatively rigid, formation of a probe-target hybrid precludes the simultaneous existence of a hybrid formed by the arm sequences. Thus, the probe undergoes a spontaneous conformational change that forces the arm sequences apart and causes the fluorophore and quencher to move away from each other. Since the fluorophore is no longer in close proximity to the quencher, it fluoresces when illuminated by light within its excitation range. The probes are termed “Molecular Beacons” because they emit a fluorescent signal only when hybridized to target molecules.

Another system for real time DNA amplification and detection is the LightCycle florescent hybridization probe analysis. In addition to the reaction components used for conventional PCR, two specially designed, sequence specific oligonucleotides labelled with fluorescent dyes are applied for this detection method. This allows highly specific detection of the amplification product as described below.

Three essential components for using florescence-labelled oligonucleotides as Hybridization probes are: two different oligonucleotides (labelled) and the amplification product. Oligonucleotide 1 carries a fluorescein label at its 3′ end whereas oligonucleotide 2 carries another label (for example, LC Red 640 or LC Red 705) at its 5′ end. The sequences of the two oligonucleotides are selected such that they hybridize to the amplified DNA fragment in a head to tail arrangement. When the oligonucleotides hybridize in this orientation, the two fluorescence dyes are positioned in close proximity to each other. The first dye (e.g., fluorescein) is excited by the LightCycler's LED (Light Emitting Diode) filtered light source, and emits green fluorescent light at a slightly longer wavelength. When the two dyes are in close proximity, the emitted energy excites the LC Red 640 or LC Red 705 attached to the second Hybridization Probe that subsequently emits red fluorescent light at an even longer wavelength. This energy transfer, referred to as FRET (Forster Resonance Energy Transfer, or Fluorescence Resonance Energy Transfer) is highly dependent on the spacing between the two dye molecules. Only if the molecules are in close proximity (a distance between 1-5 nucleotides) the energy is transferred at high efficiency. Choosing the appropriate detection channel, the intensity of the light emitted by the LC Red 640 or LC Red 705 is filtered and measured by optics in the thermocycler. The increasing amount of measured fluorescence is proportional to the increasing amount of DNA generated during the ongoing amplification process. Since LC Red 640 and LC Red 705 only emit a signal when both oligonucleotides are hybridized, the fluorescence measurement is performed after the annealing step. Using hybridization probes can also be beneficial if samples containing very few template molecules are to be examined. DNA-quantification with hybridization probes is not only sensitive but also highly specific. It can be compared with agarose gel electrophoresis combined with Southern blot analysis but without all the time consuming steps which are required for the conventional analysis.

A number of real time fluorescent detection thermocyclers are currently available with the chemistries being interchangeable with those discussed above as the final product is emitted fluorescence. Such thermocyclers include the Applied Biosystems PRISM 7700, Corbett Research's Rotogene, the Hoffman La Roche Light Cycler, and the iCycler produced by Bio-Rad. It is envisaged that any of the above thermocyclers could be adapted to perform the methods of the present invention.

Reaction mixes of the present invention can include control primers as well as the amplification primers. Controls can test for the function of both the amplification and the sequence specific nucleic acid cleavage agent to prevent false negative and false positive results. Primers for the amplification control amplicon are designed to amplify any locus (X) that is devoid of the recognition sequence of the sequence specific nucleic acid cleavage agent used for the present invention. The presence of these amplicons indicates that all reaction components and conditions were adequate for amplification. The primers for the sequence specific nucleic acid cleavage agent control can hybridize to any locus (Y) and are designed to induce the recognition sequence of the cleavage agent in all amplicons. These primers will only amplify when the cleavage activity of the cleavage agent is insufficient to inhibit amplification of these control amplicons. When REMS-PCR products are to be analysed by gel electrophoresis, the reaction must be stopped at a point before wild type (and RE control) amplicons reach a threshold amount whereby they become visible by EtBr staining. This generally occurs at between 30 and 35 cycles and hence reactions are usually stopped at around 30 cycles. When the reactions are monitored in real time (eg on the ABI PRISM 7700) it is no longer necessary to terminate the reaction prior to amplification of the wild type and sequence specific nucleic acid cleavage agent control amplicons.

In the example described below, only K-ras diagnostic primers and one target nucleic acid were included; both the amplification control and sequence specific nucleic acid cleavage agent control primers were omitted. In a multiplex assay of the present invention using REMS and DzyNA systems, both diagnostic and control amplicons could be generated using DzyNA primers. Reactions could include multiple substrates, each of which is specific for a specific type of amplicon (diagnostic or control), and each of which could be labelled with a different reporter fluorophore. In this format, individual amplicons could be analysed simultaneously in a single reaction. The relative Ct values observed in the presence of the sequence specific nucleic acid cleavage agent will reflect the percentage of the template that contains the cleavage recognition site (Table 1) provided that a) the efficiencies of amplification of the target sequences (Diagnostic locus and locus X) lacking the cleavage recognition sites are equal for the diagnostic and amplification control primers and b) efficiencies of amplification of the target sequences (Diagnostic locus and locus Y) containing cleavage recognition sites are equal for the diagnostic and sequence specific nucleic acid cleavage agent control amplicons.

TABLE 1

| | % of alleles | | |
|---|---|---|---|
| | with a | | Relative Ct in real time |
| Locus | cleavage site | Function | (where Ct 1 < Ct 2) |
| X | 0% | amplification | Ct = 1 |
| | | control | |
| Diagnostic | Unknown | Diagnostic | Mutant 1 ≦ Ct < 2 |
| | | eg K-ras | Wild type Ct = 2 |
| Y | 100% | cleavage | Ct = 2 |
| | | agent control | |

In real time the amplification control would amplify first and the sequence specific nucleic acid cleavage agent control amplicon would amplify last. Diagnostic amplicons will reach the threshold level of fluoresence at the same time (Ct) as the amplification control when the starting template is 100% mutant. Diagnostic amplicons would reach the threshold level of fluoresence at the same time (Ct) as the sequence specific nucleic acid cleavage agent control when the starting template is 100% wild type. In reactions containing a mixture of mutant and wild type molecules, diagnostic amplicons would reach the threshold level of fluoresence at a time point that is intermediate to that observed for the amplification control and sequence specific nucleic acid cleavage agent control amplicons. Unambiguous detection of point mutations using real time analysis could be achieved by simultaneously monitoring diagnostic, amplification control and sequence specific nucleic acid cleavage agent control amplicons in a multiplex system.

The efficiency of amplification of specific amplicons in multiplex systems can be easily adjusted by one skilled in the art by altering primer length, relative primer concentrations and other reaction conditions (buffer, temperature profile etc). Multiplex homogeneous amplification and detection systems would be tolerant to minor differences in the efficiency of amplification of the diagnostic, amplification control and sequence specific nucleic acid cleavage agent control amplicons. The efficiency of amplification of the diagnostic primers must be greater than, or equal to, that of the amplification control primers when both their respective target sequences lack cleavage recognition sites, and the efficiency of amplification of the diagnostic primers must be less than, or equal to, that of the sequence specific nucleic acid cleavage agent control primers when both their respective target sequences contain cleavage recognition sites. In reactions, where the amplification efficiencies of the diagnostic and control amplicons are not equal, the Ct values can still be used as a marker of specific sequences associated with the presence or absence of a cleavage recognition site (Table 2).

TABLE 2

| | % of alleles | | |
|---|---|---|---|
| | with a | | Relative Ct in real time |
| Locus | cleavage site | Function | (where Ct 1 < Ct 2) |
| X | 0% | amplification | Ct = 1 |
| | | control | |
| Diagnostic | Unknown | Diagnostic | Mutant Ct < 2 |
| | | eg K-ras | Wild type Ct ≧ 2 |
| Y | 100% | cleavage | Ct = 2 |
| | | agent control | |

When REMS PCR reactions are analysed by gel electrophoresis the result can only be positive or negative for the presence of a specific sequence (mutant or wild type) at the locus being examined. Hence, identification of a heterozygous individual would require two reactions (one targeting the mutant allele and a second targeting the wild type allele). However, in real time determination of the homozygous mutant, heterozygous and homozygous wild type genotypes can be achieved in a single multiplex reaction (Table 3). The amplicon which serves as a amplification control could function as a marker for homozygous mutant genotypes; the cleavage agent control could function as a marker for homozygous wild type genotypes; and heterozygous genotypes would have a Ct value which are intermediate between the homozygous mutant and wild type markers. Unambiguous genotyping using real time analysis requires a multiplex system, which incorporates diagnostic, homozygous mutant control and homozygous wild type control amplicons, and a platform which is capable of simultaneously monitoring three reporter dyes.

TABLE 3

| | % of | | |
|---|---|---|---|
| | alleles | Genotyping using the present invention: | |
| | with a | Homozygous mutant (M); Heterozygous (M:WT); | |
| | cleavage | Homozygous wild type (WT) | |
| Locus | site | Function | Real time (Ct 1 < Ct 2) |
| X | 0% | M marker | Ct = 1 |
| Diagnostic | Unknown | Diagnostic eg | M Ct ≦ 1 |
| | | Haemachromatosis | 1 < M:WT Ct < 2 |
| | | | WT Ct ≧ 2 |
| Y | 100% | WT marker | Ct = 2 |

The present invention can be used for the analysis of either acquired or inherited genetic polymorphisms (eg point mutations, small deletions or insertions) or epi-genetic polymorphisms (eg aberrantly methylated cytosines). In the latter case, the method of the present invention can be used to analyse bisulfite induced polymorphisms which reflect the presence of methylated or unmethylated cytosines in the original (untreated) genomic DNA template. Bisulfite treatment of genomic DNA converts cytosine (C) to uracil (U), whereas 5-methylcytosine (^{m}C) is resistant to modification (Frommer et al., 1992). Cleavage by the sequence specific nucleic acid cleavage agent allows the determination of the methylation status of the original template. For example, BstU I can be used to confirm the presence of methylated sequence (^{m}C) at a locus that results in protection of the template from bisulfite modification. The presence or absence of methylated cytosines can be used as a marker for tumour cells, foetal cells or pathogens.

The method of the present invention can be used to detect hypermethylated sequences within the promoter region of genes in association with human tumours. For instance, hypermethylation of the CpG island in the E-cadherin gene promoter has been detected in breast, prostate, colon, bladder, and liver tumours. Other examples of hypermethylation of genes associated with human tumours include p16 (lung, breast, colon, prostate, renal, liver, bladder, and head and neck tumours), the von Hippel Lindau (VHL) gene (renal cell tumours), BRCA1 (breast tumours), p15 (leukemias, Burkitt lymphomas), hMLH1 (colon tumours), ER (breast, colon, lung tumours; leukemias), HIC1 (brain, breast, colon, renal tumours), MDG1 (breast tumours), GST-π (prostate tumours), O⁶-MGMT (brain tumours), calcitonin (carcinoma, leukemia), and myo-D (bladder tumours). In one embodiment, the present invention is designed to detect methylated sequences, the bisulphite treated unmethylated sequences, but not the bisulphite treated methylated sequences, contain the recognition sequence for a sequence specific nucleic acid cleavage agent. Amplification of sequences derived from unmethylated sequences is inhibited by the activity of the cleavage agent. In contrast, methylated sequences are selectively amplified by the polymerase during the amplification.

Methods of the present invention can also be designed to selectively inhibit amplification of methylated but not unmethylated sequences. If protocols for the present invention are designed to detect unmethylated sequences, the bisulfite treated methylated sequences but not the bisulfite treated unmethylated sequences contain the recognition sequence for a sequence specific nucleic acid cleavage agent. Hypomethylation is associated with transcriptional activation of genes such as urokinase or S100A4 in cancer.

Primers for this aspect of the present invention can be chosen such that they will selectively amplify nucleic acid that has efficiently reacted with bisulfite by designing primers to anneal to sequences containing U in place of C, and by choosing sequences that originally contained several Cs. The primers used are chosen so that they do not span CpG dinucleotides and hence do not differentially anneal to templates according to their original methylation status. This ensures that the amplified product is not the result of mispriming from alternatively modified templates containing U instead of C or visa versa.

The limits of detection of the present invention allows detection of sequence polymorphisms present in a 1,000 fold excess of wild type sequences. The literature suggests that this level of sensitivity will be adequate for analysis of genetic mutations (eg K-ras) in DNA extracted from clinical specimens including tissue resections and biopsies, cytology samples and body fluids/excretions such as stools, urine and sputum containing small numbers of exfoliate tumour cells (Sidransky et al., 1992; Mao et al. 1994). Hypermethylated sequences have been detected in normal and tumour tissue, (Wong et al., 1999), paraffin embedded tissues (Xiong and Laird, 1997), as well as plasma and serum using bisulfite/PCR protocols, which have equivalent or lesser sensitivity than the methods of the present invention.

Differences in patterns of covalent modification of nucleotide bases at discreet genetic loci could be used as a marker of disease states such as fragile X syndrome, altered gene imprinting states, and cancer. The selective nature of nucleic acid amplification means that it is well suited to analysis of rare genetic variations eg tumour sequences in a background of normal sequences, or foetal sequences in a background of maternal sequences. The technology could form the basis of minimally invasive assays in which body fluids are analysed for the presence of variant sequences.

The present invention provides a sensitive, rapid method that is suitable for analysis of genetic and epi-genetic variations that are associated with disease. The ability to simultaneously sustain the activities of a sequence specific nucleic acid cleavage agent and a polymerase during amplification allows the development of simple protocols for selective amplification of variant sequences in reactions that contain all reagents, including all enzymes, at the initiation of the amplification. Reactions can be performed in a closed system that reduces the opportunity for contamination during amplification. In general, the reactions do not require further manipulation prior to detection, however, the method does not preclude subsequent analysis of diagnostic amplicons for identification of the exact nucleotide substitution. A reduction in the number of steps required for selective amplification and analysis with the sequence specific nucleic acid cleavage agent makes the present assay rapid, less labour intensive and more amenable to automation.

The present invention overcomes at least some of the current limitations of REMS-PCR. The method of the present invention can be used for the analysis of a range of genetic polymorphisms including point mutations, small deletions and insertions. In the present invention, the ability, or inability, of a sequence specific nucleic acid cleavage agent to induce temporal inhibition in amplification is used as a marker of the presence or absence of a specific polymorphism in the target nucleic acid. The present invention provides methods suitable for analysis of inherited polymorphisms by facilitating simultaneous detection of both the homozygous and heterozygous states in a single reaction. Furthermore, when the target is an acquired mutation, the invention abrogates the need to terminate the reactions prematurely before rare mutant alleles have amplified and this is likely to increase the sensitivity and reliability of analysis. Analysis in real time means that ambiguous results are not obtained (ie as occurred when both diagnostic and sequence specific nucleic acid cleavage agent control amplicons were simultaneously visualized by electrophoresis). Finally the method is faster and simpler since it overcomes the need for post-amplification analysis by methods such as electrophoresis. This closed system also eliminates the potential for false positives associated with contamination by amplicons from other reactions. The invention is well suited for analysis of specimens in clinical laboratories.

This invention will be better understood by reference to the Experimental Details which follow, but those skilled in the art will readily appreciate that the specific experiments detailed are only illustrative of the invention.

### EXAMPLE

Assay for Detecting Sequence Polymorphisms in the K-ras Gene

Methods

PCR Primers. The 5′ PCR primer 5KIT (5′-TATAAACTTGTGGTAGTTGGACCT-3′) contains sequence which is complementary to the human K-ras gene (underlined). A single mismatched base located near the 3′end of 5KIT results in the induction of a recognition/cleavage site for the thermostable RE BstN I in PCR amplicons provided the first two bases in codon 12 of the K-ras gene are wild type (GG). The 3′ primer 3K45Dz2 (5′-CCACTCTCGTTGTAGCTAGCCTATTAGCTGTATCGTCAAGCCACTCTTGC-3′) is a DzyNA-PCR primer which contains (a) a 5′ region containing the catalytically inactive antisense sequence complementary to an active 10:23 deoxyribozyme (plain bold text indicates the complement of the arms that hybridise to the reporter substrate, italic bold text indicates the complement of the 10-23 catalytic domain) and (b) a 3′ region which is complementary to the human K-ras gene (underlined). Primers were synthesized by Macromolecular Resources (USA) or Pacific Oligos (Lismore NSW Australia).

Reporter Substrates. The DzyNA reporter substrate SubDz2 (5′-CCACTCguATTAGCTGTATCGTCAAGCCACTC-3′) is a chimeric oligonucleotide containing both RNA (lower case) and DNA bases. The substrate is designed such that the bond between the gu ribonucleotides is cleaved by active deoxyribozymes generated during DzyNA-PCR. The substrate was synthesized with a reporter 6-carboxyfluorescein (FAM) at the 5′ end and a quencher 6-carboxytetramethylrhodamine (TAMRA) incorporated internally at nucleotide 10. A 3′-phosphate group was added to prevent extension by DNA polymerase during PCR. The substrate was synthesized by Oligos Etc., Inc. (Wilsonville, Oreg., USA).

DNA templates for PCR. The plasmid pCRKM and pCRKW contained the genomic sequence between nucleotides 84 and 289 of the human cellular c-Ki-ras2 gene, exon 1 (GenBank Locus HUMRASK02, Accession number L00045) cloned into the vector pCR2.1 (Original TA cloning kit, Invitrogen). The sequence at codon 12 is mutant (GTT) in pCRKM and is wild type (GGT) in pCRKW. Plasmid was purified by column chromatography (Qiaprep Spin Plasmid kit, Qiagen) and used as template in PCR reactions.

Amplification and detection. PCR was performed using the 5′ REMS primer 5KIT and the 3′ DzyNA primer 3K45Dz2 to facilitate amplification of K-ras. All amplicons generated during PCR contain active deoxyribozymes at their 3′ termini, however, only those with wild type sequence at codon 12 will contain BstN I RE sites near their 5′ termini. The reaction mixes contained 0.4 mM 5KIT, 0.06 mM 3K45Dz2, 0.2 mM SubDz2, 1×HTris 50 buffer (100 mM NaCl, 50 mM Tris HCl pH 8.3 at 25° C.), 4 mM MgCl₂, 40 U of BstN I and 3 Units AmpliTaq DNA polymerase (PE Biosystems) preincubated with TaqStart™ antibody (Clontech) in the ratio 1:10 according to manufacturers instructions. Duplicate reactions contained equal amounts (10⁵ copies) of plasmid DNA that was either mutant (GTT), or wild type (GGT), or mixtures at a ratio of 1:1 or 1:10 mutant to wild type. The thermocycling profile used was 94° C. for 3 min, 10 cycles of 65° C. for 1 min, 94° C. for 20 s, and 60 cycles of 40° C. for 30 s, 60° C. for 30 s and 94° C. for 20 s. Amplification was performed on the ABI PRISM™ 7700 SDS and the DzyNA REMS-PCR reactions were monitored in real time.

Data analysis. ABI PRISM™ 7700 SDS software was used to analyse the increase in FAM fluorescence at 530 nm following cleavage of substrate by amplicons harbouring active deoxyribozymes. A cycle threshold value (Ct) was determined for each sample corresponding to the cycle when fluorescence exceeded a defined baseline signal (threshold ΔRn) within the log phase of product accumulation. The SDS software analysis was performed in the absence of correction for the passive reference ROX as this was not included in the DzyNA-REMS-PCR mixes.

Results and Discussion

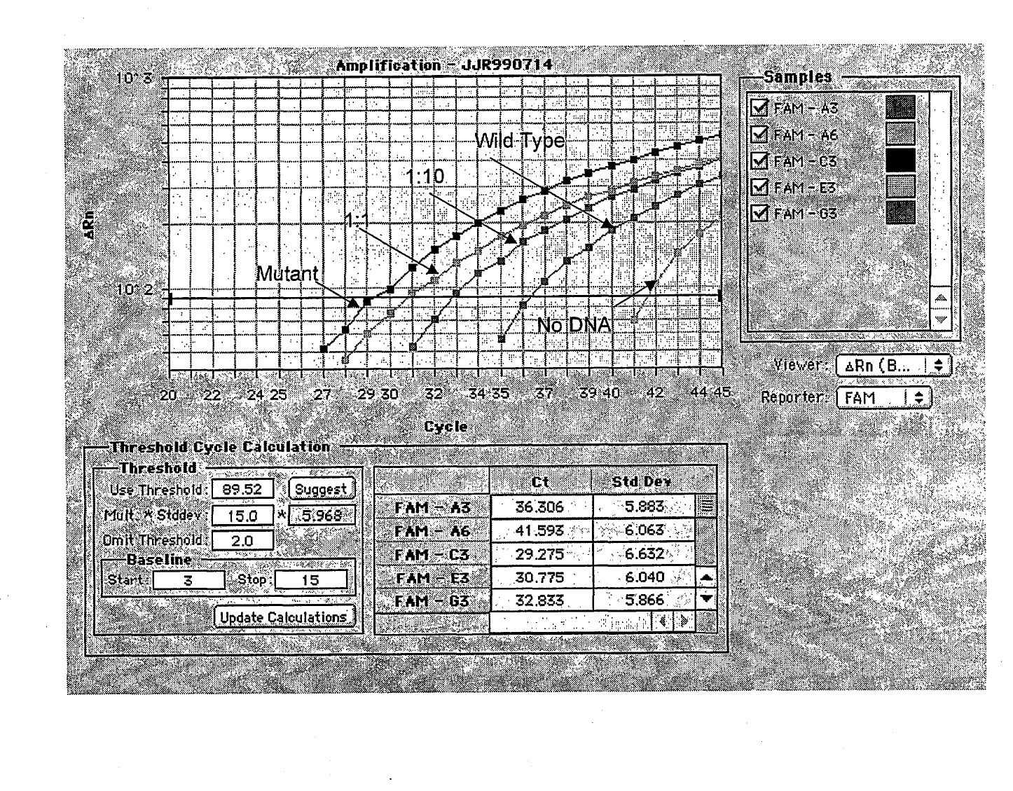

Although all reactions contained equal amounts of plasmid DNA (by weight), in real time the reactions reached the threshold value in the following order; Mutant (Ct=29), 1:1 Mutant: Wild type (Ct=31), 1:10 Mutant: Wild type (Ct=33) and Wild type (Ct=36) (FIG. 1). The addition of BstN I caused temporal inhibition of amplification (delay in reaching threshold ΔRn) of wild type but not mutant sequences. The delay in reaching threshold ΔRn resulted in higher Ct values for reactions containing wild type template compared to reactions containing an equivalent amount of mutant template. Reactions containing equal amounts of template that consisted of mixtures of mutant and wild type sequences had Ct values that were intermediate between the Ct observed for reactions containing only mutant template and those observed for reactions containing only wild type templates.

In conclusion, the present invention was used for the analysis of sequence variations at codon 12 of the human K-ras gene. Reactions were monitored in real time using the DzyNA-PCR strategy. The presence of a RE recognition site in wild type amplicons resulted in temporal inhibition of amplification of these sequences. This temporal inhibition was reflected by an increase in the Ct values observed in reactions containing only wild type template compared to reactions containing only mutant template. In reactions containing equal amounts of template, the lowest Ct value was observed in reactions containing only mutant template and the highest Ct value was observed in reactions containing only wild type template only. Intermediate Ct values were observed in reactions containing a mixture of mutant and wild type template with the observed Ct value increasing as the ratio of wild type to mutant molecules increased.

The disclosure of all references referred to herein are incorporated herein by cross reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Antonarakis, S. E. (1989) N Engl J Med, 320, 153-163 [published erratum appears in N Engl J Med Jul. 6, 1989;321(1):56].

Breaker, R. R. (1997) Nat Biotechnol, 15, 427-431.

Breaker, R. R. and Joyce, G. F. (1994) Chem Biol, 1, 223-229.

Cairns, M. J., King, A. and Sun, L -Q. (2000) Nucleic Acids Res, 28, e9 i-vi.

Carmi, N., Shultz, L. A. and Breaker, R. R. (1996) Chem Biol, 3, 1039-1046.

Chehab, F. F., Doherty, M., Cai, S. P., Kan, Y. W., Cooper, S. and Rubin, E. M. (1987) Nature, 329, 293-294 [published erratum appears in Nature Oct. 22, 1987-28;329(6141):678].

Cohen, J. B. and Levinson, A. D. (1988) Nature, 334, 119-124.

Compton, J. (1991) Nature, 350, 91-92.

Cuenoud, B. and Szostak, J. W. (1995) Nature, 375, 611-614.

Fahy, E., Kwoh, D. Y. and Gingeras, T. R. (1991) PCR Methods Appl, 1, 25-33.

Frommer, M., McDonald, L. E., Millar, D. S., Collis, C. M., Watt, F., Grigg, G. W., Molloy, P. L. and Paul, C. L. (1992) Proc Natl Acad Sci USA, 89, 1827-1831.

Fuery, C. J., Imprey, H. L., Roberts, N. J., Applegate, T. L., Ward, R. L., Hwakins, N. J., Sheehan, C. A., O'Grady, R. and Todd, A. V. (2000) Clin Chem, 46, 620-624.

Gingeras, T. R., Whitfield, K. M. and Kwoh, D. Y. (1990) Ann Biol Clin (Paris), 48, 498-501.

Haseloff, J. and Gerlach, W. L. (1988) Nature, 334, 585-591.

Heid, C. A., Stevens, J., Livak, K. J. and Williams, P. M. (1996) Genome Res, 6, 986-994.

Illangasekare, M., Sanchez, G., Nickles, T. and Yarus, M. (1995) Science, 267, 643-647.

Impey, H. L., Applegate, T. L., Haughton, M. A., Fuery, C. J., King J. E. and Todd, A. V. (2000) Analytical Biochemistry, 286, 300-303.

Jonas, V., Alden, M. J., Curry, J. I., Kamisango, K., Knott, C. A., Lankford, R., Wolfe, J. M. and Moore, D. F. (1993) J Clin Microbiol, 31, 2410-2416.

Joyce, G. F. (2000) RNA cleavage by the 10:23 DNA enzyme. Methods in Enzymology (in press).

Kramvis, A., Bukofzer, S. and Kew, M. C. (1996) J Clin Microbiol, 34, 2731-2733.

Kwok, S., Kellogg, D. E., McKinney, N., Spasic, D., Goda, L., Levenson, C. and Sninsky, J. J. (1990) Nucleic Acids Res, 18, 999-1005.

Lee, L. G., Connell, C. R. and Bloch, W. (1993) Nucleic Acids Res, 21, 3761-3766.

Leone, G., van Schijndel, H., van Gemen, B., Kramer, F. R. and Schoen, C. D. (1998) Nucleic Acids Res, 26, 2150-2155.

Levi, S., Urbano-Ispizua, A., Gill, R., Thomas, D. M., Gilbertson, J., Foster, C. and Marshall, C. J. (1991) Cancer Res, 51, 3497-3502.

Li, Y. and Sen, D. (1996) Nat Struct Biol, 3, 743-747.

Livak, K. J., Floof, S. J. A., Marmaro, J., Giusti, W. and Deetz, K. (1995) PCR Methods and Applic, 4, 357-362.

Liu, Y. S., Thomas, R. J. and Phillips, W. A. (1995) Nucleic Acids Res, 23, 1640.

Lohse, P. A. and Szostak, J. W. (1996) Nature, 381, 442-444.

Mao, L., Hruban, R. H., Boyle, J. O., Tockman, M. and Sidransky, D. (1994) Cancer Res, 54, 1634-1637.

Nazarenko, I. A., Bhatnagar, S. K. and Hohman, R. J. (1997) Nucleic Acids Res, 25, 2516-2521.

Perriman, R., Delves, A. and Gerlach, W. L. (1992) Gene, 113, 157-163.

Railard, S. A. and Joyce, G. F. (1996) Biochemistry, 35, 11693-11701.

Saiki, R. K., Scharf, S., Faloona, F., Mullis, K. B., Horn, G. T., Erlich, H. A. and Arnheim, N. (1985) Science, 230, 1350-1354.

Sambrook, J., Fritsch, E. F. and Maniatis, T. (1989) Molecular Cloning: A Laboratory Manual. Cold Spring Harbour Laoratory Press, New York.

Santoro, S. W. and Joyce, G. F. (1997) Proc Natl Acad Sci USA, 94, 4262-4266.

Santoro, S. W. and Joyce, G. F. (1998) Biochemistry, 37, 13330-13342.

Shippy, R., Lockner, R., Farnsworth, M. and Hampel, A. (1999) Molecular Biotechnology, 12, 117-129.

Sidransky, D., Tokino, T., Hamilton, S. R., Kinzler, K. W., Levin, B., Frost, P. and Vogelstein, B. (1992) Science, 256, 102-105.

Tarasow, T. M., Tarasow, S. L. and Eaton, B. E. (1997) Nature, 389, 54-57.

Todd, A. V., Fuery, C. J., Impey, H. L., Applegate, T. L. and Haughton, M. A. (2000) Clin Chem, 46, 625-630.

Todd, A. V., Ireland, C. M. and Iland, H. J. (1991a) Leukemia, 5, 160-161.

Todd, A. V., Ireland, C. M., Radloff, T. J., Kronenberg, H. and Iland, H. J. (1991b) Am J Hematol, 38, 207-213.

Tyagi, S. and Kramer, F. R. (1996) Nat Biotechnol, 14, 303-308.

Walder, R. Y., Hayes, J. R. and Walder, J. A. (1993) Nucleic Acids Res, 21, 4339-4343.

Walker, G. T., Fraiser, M. S., Schram, J. L., Little, M. C., Nadeau, J. G. and Malinowski, D. P. (1992) Nucleic Acids Res, 20, 1691-1696.

Ward, R., Hawkins, N., O'Grady, R., Sheehan, C., O'Connor, T., Impey, H., Roberts, N., Fuery, C. and Todd, A. (1998) Am J Pathol, 153, 373-379.

Watson, J. D., Tooze, J. and Durtz, D. T. (1983) Recombinant DNA: A short course. Scientific American Books, New York.

Wittwer, C. T., Herrmann, M. G., Moss, A. A. and Rasmussen, R. P. (1997) Biotechniques, 22, 130-131, 134-138.

Wong, I. H., Lo, Y. M., Zhang, J., Liew, C. T., Ng, M. H., Wong, N., Lai, P. B., Lau, W. Y., Hjelm, N. M. and Johnson, P. J. (1999) Cancer Res, 59, 71-73.

Xoing, Z. and Laird, P. W. (1997) Nucleic Acids Res, 15, 2532-2534.

1. A method of detecting a genetic polymorphism in an individual or between individuals, the method comprising the following steps:
(1) obtaining a sample containing nucleic acid from an individual;
(2) contacting the sample, under conditions which permit primer-initiated nucleic acid amplification and nucleic acid cleavage, with
(i) a primer suitable for initiating amplification,
(ii) an indicator system which provides a signal proportional to the amount of amplification product, and
(iii) a sequence specific nucleic acid cleavage agent; and
(3) measuring the signal produced by the indicator system against time;
wherein cleavage of the amplification product by the cleavage agent results in a delay in the accumulation of amplification product comprising the sequence recognised by the cleavage agent relative to the accumulation of the amplification product not comprising the sequence recognised by the cleavage agent.

2. The method according to claim 1, wherein the primers are designed such that they induce the sequence recognised by the sequence specific nucleic acid cleavage agent into the nucleic acid resulting from amplification of the sample nucleic acid not including the polymorphism.

3. The method according to claim 1, wherein the primers are designed such that they induce the sequence recognised by the sequence specific nucleic acid cleavage agent into the nucleic acid resulting from amplification of the sample nucleic acid including the polymorphism.

4. The method according to any one of claims 1 to 3, wherein the sequence specific nucleic acid cleavage agent is a thermostable restriction endonuclease.

5. The method according to claim 4, wherein the thermostable restriction endonuclease is selected from the group consisting of Bst NI, Bsl I, Tru 9I, Tsp 509 I, Tsp 45 I, Tth 111 I, Tsp RI, Tse I, Tfi I, Sml I, Bso B I, Bst E II, Psp G I, Bst F5 I, and Sfi I.

6. The method according to any one of claims 1 to 3, wherein the sequence specific nucleic acid cleavage agent is a catalytic nucleic acid.

7. The method according to claim 6, wherein at least one primer comprises a region which binds to the sample nucleic acid and a region which is an antisense sequence of the catalytic nucleic acid such that on amplification the catalytic nucleic acid is produced.

8. The method according to claim 6 or claim 7, wherein the catalytic nucleic acid is selected from the group consisting of ribozymes and deoxyribozymes.

9. The method according to any one of claims 1 to 8, wherein the signal produced by the indicator system is fluorescence.

10. The method according to claim 9, wherein the indicator system comprises a catalytic nucleic acid and a substrate, the substrate comprising a fluorophore and a molecule that quenches fluorescence from the fluorophore separated by a site cleavable by the catalytic nucleic acid, wherein the primers are designed such that the amplification products comprise the catalytic nucleic acid.

11. The method according to claim 10, wherein one primer comprises a region which binds to the nucleic acid and a region which is an antisense sequence of the catalytic nucleic acid.

12. The method according to any one of claims 1 to 9, wherein the indicator system comprises the TaqMan™ nucleic acid detection system.

13. The method according to any one of claims 1 to 9, wherein the indicator system comprises the Molecular Beacon™ nucleic acid detection system.

14. The method according to any one of claims 1 to 9, wherein the indicator system comprises the Hybridisation Probe nucleic acid detection system.

15. The method according to any one of claims 1 to 9, wherein the indicator system comprises the Sunrise™ nucleic acid detection system.

16. The method according to any one of claims 1 to 15, wherein the nucleic acid is a DNA.

17. The method according to any one of claims 1 to 15, wherein the nucleic acid is an RNA molecule, and step (2) further comprises the step of first reverse transcribing the RNA sequence to DNA.

18. The method according to any one of claims 1 to 17, wherein the amplification is performed by a polymerase chain reaction (PCR).

19. The method according to any one of claims 1 to 17, wherein the amplification is performed by a strand displacement amplification assay (SDA).

20. The method according to any one of claims 1 to 17, wherein the amplification is performed by a transcription-mediated amplification reaction (TMA).

21. The method according to any one of claims 1 to 17, wherein the amplification is performed by a self-sustained sequence replication amplification reaction (3SR).

22. The method according to any one of claims 1 to 17, wherein the amplification is performed by a nucleic acid sequence replication based amplification reaction (NASBA).

23. The method according to any one of claims 1 to 22, wherein the genetic polymorphism is within a gene selected from the group consisting of; ras proto-oncogenes (K-ras, N-ras, and H-ras), p53 tumour suppressor gene, a HIV-I gene, haemocromatosis, cystic fibrosis trans-membrane conductance regulator, α-antitrypsin, Factor V and β-globin.

24. The method according to any one of claims 1 to 23, wherein the genetic polymorphism is in codon 12 of K-ras.

25. A method of detecting an epi-genetic polymorphism in an individual or between individuals, the method comprising the following steps:
(1) obtaining a sample containing nucleic acid from an individual;
(2) reacting the nucleic acid from step (1) with a compound that differentially modifies nucleotide bases depending on whether the specific base contains, or lacks, a covalent modification;
(3) contacting the nucleic acid from step (2), under conditions which permit primer-initiated nucleic acid amplification and nucleic acid cleavage, with
(i) a primer suitable for initiating amplification,
(ii) an indicator system which provides a signal proportional to the amount of amplification product, and
(iii) a sequence specific nucleic acid cleavage agent; and
(4) measuring the signal produced by the indicator system against time;
wherein cleavage of the amplification product by the cleavage agent results in a delay in the accumulation of amplification product comprising the sequence recognised by the cleavage agent relative to the accumulation of the amplification product not comprising the sequence recognised by the cleavage agent.

26. The method according to claim 25, wherein the primers are designed such that they induce the sequence recognised by the sequence specific nucleic acid cleavage agent into the nucleic acid resulting from amplification of the sample nucleic acid not including the polymorphism.

27. The method according to claim 25, wherein the primers are designed such that they induce the sequence recognised by the sequence specific nucleic acid cleavage agent into the nucleic acid resulting from amplification of the sample nucleic acid including the polymorphism.

28. The method according to any one of claims 25 to 27, wherein the covalent modification is methylation of a base.

29. The method according to any one of claims 25 to 28, wherein the nucleic acid is reacted with bisulphite.

30. The method according to any one of claims 25 to 29, wherein the sequence specific nucleic acid cleavage agent is a thermostable restriction endonuclease selected from the group consisting of Bst N I, Psp G I, Bsl I, Tru9 I, Bst U I and Tsp509 I.

31. The method according to any one of claims 25 to 30, wherein the epi-genetic polymorphism is within the promoter region of a gene associated with human tumours.

32. The method according to claim 31, wherein the promoter region is from a gene selected from the group consisting of: p16, E-cadherin, the von Hippel Lindau (VHL) gene, BRCA1, p15, hMLH1, ER, HIRC1, MDG1, GST-π, O⁶-MGMT, calcitonin, urokinase, S10A4, and myo-D.

33. The method according any one of claims 1 to 32 in which the endonuclease activity of the thermostable restriction endonuclease decreases throughout the amplification process.

34. The method according to any one of claims 1 to 33, wherein the sample is obtained from a mammal.

35. The method according to claim 34, wherein the mammal is a human.

36. The method according to any one of claims 1 to 35, wherein the method is performed in a closed vessel or chamber.


SEQUENCE LISTING (3 sequences)

SEQ ID NO: 1 - DNA, 24 bp, Artificial
  PCR primer
tataaacttgtggtagttggacct

SEQ ID NO: 2 - DNA, 48 bp, Artificial
  PCR primer
ccactctcgttgtagctagcctattagctgtatcgtcaagccactctt

SEQ ID NO: 3 - DNA, 32 bp, Artificial
  oligonucleotide
ccactcguattagctgtatcgtcaagccactc